(19) United States
(12) Patent Application Publication
(10) Pub. No.: US 2024/0374211 A1
(43) Pub. Date: Nov. 14, 2024

(54) WEARABLE HAND MOTION AND FORCE TRACKING AND FEEDBACK SYSTEM

(71) Applicant: ANSELL LIMITED, Richmond, Victoria (AU)

(72) Inventors: James Patrick THOMPSON, Pendleton, SC (US); Akilan Yaminah WALTON, Charlotte, NC (US); Stephen Dalton PITTMAN, Brookline, MA (US); Elisabeth Anne BRANHAM, Ashburnham, MA (US); Ronald MARTINEZ, Mercer Island, WA (US); Itmenon TOWFEEQ, Central, SC (US); Praveen NEDUMPULLY GOVINDAN, Atlanta, GA (US); Shridar Chandrashekhar KULKARNI, Clemson, SC (US)

(21) Appl. No.: 18/569,027

(22) PCT Filed: Jun. 15, 2022
(86) PCT No.: PCT/AU2022/050590

§ 371 Date: Dec. 11, 2023

Related U.S. Application Data
(60) Provisional application No. 63/210,628, filed on Jun. 15, 2021.

(51) Int. Cl. A61B 5/00; A61B 5/11; G09B 19/00

(52) U.S. Cl. CPC A61B 5/6824; A61B 5/1121; A61B 5/1126; A61B 5/486; G09B 19/003

(57) ABSTRACT

A wearable system (100) for tracking hand and wrist motion and forces includes a wearable pod (102) configured to be worn on a wrist or a back of a hand. the pod including: a sensor (104) configured to output a movement and an orientation measurement of the wrist or hand: a haptic feedback device (106) configured to provide sensory feedback to the hand or wrist: a processor (108) in communication with the at least one sensor and the haptic feedback device. the processor configured to determine whether the hand is in a neutral position based on the movement and orientation measurement of the wrist or hand and output a haptic feedback signal to the haptic feedback device based on the determination of whether the hand is in a neutral position: and a memory in communication with the processor and configured to record measurements from the sensors as measurement data.

### BACKGROUND

### Field

Embodiments of the present invention relate generally to protective articles and, more particularly, to wearable hand motion and feedback systems.

### Description of the Related Art

Gloves are used in many industries and in households. Many activities are of a repetitive nature, which can cause or exacerbate repetitive motion injuries, such as lateral epicondylitis and carpal tunnel syndrome and musculo-skeletal disease. Also, the longer a person engages in activities using the hand, the more tired the hand can become.

Hand and wrist Work-related Musculoskeletal Disorders (WMSDs) represent a substantial proportion of work-related injuries and are associated with relatively high medical costs and loss of work. Repetitive tasks of the hand have a high-risk of hand disorders, namely carpal tunnel syndrome and wrist tendinopathy.

### SUMMARY

A wearable system for tracking hand and wrist motion, and providing corrective feedback to prevent injuries and WMSDs, substantially as shown and described in connection with at least one of the figures, as set forth more completely in the claims, is provided. Various advantages, aspects and novel and inventive features of the present disclosure, as well as details of illustrative embodiments thereof, will be more fully understood from the following description and drawings. The foregoing summary is not intended, and should not be contemplated, to describe each embodiment or every implementation of the embodiments. Other and further embodiments of the present disclosure are described below.

In accordance with one aspect, a wearable system for tracking hand and wrist motion includes a wearable motion pod configured to be worn on a a wrist or a hand, the motion pod including: a sensor configured to output an orientation and movement measurement of the wrist or hand: a haptic feedback device configured to provide sensory feedback to the wrist or hand: a processor in communication with the at least one sensor and the haptic feedback device, the processor configured to determine whether the hand is in a neutral position based on the orientation and movement measurement of the wrist or hand and output a haptic feedback signal to the haptic feedback device based on the determination of whether the hand is in a neutral position: and a memory in communication with the processor and configured to record measurements from the sensors as measurement data.

According to another aspect, a method for tracking hand and wrist motion includes i) measuring an orientation and movement of a wrist or hand of a user, ii) determining from the measuring in i) whether the hand is in a neutral position, iii) incrementing a compliance time if the hand is in a neutral position and incrementing a non-compliance time if the hand is not in a neutral position: determining whether a measurement time window has expired: if the measurement time window has not expired, repeating i), ii), and iii), and if the time window has expired, determining whether a total non-compliance time is greater than a threshold; and if the total non-compliance time is greater than the threshold, generating a haptic feedback alert.

According to yet another aspect, a wearable motion pod configured to be worn on a wrist or a hand includes a plurality of sensors configured to output a movement and orientation measurement of the wrist or hand, the plurality of sensors including an accelerometer and a gyroscope,: a haptic feedback device configured to provide sensory feedback to the wrist or hand: a processor in communication with the sensor and the haptic feedback device, the processor configured to determine whether the hand is in a neutral position based on the movement and orientation measurement of the wrist or hand and output a haptic feedback signal to the haptic feedback device based on the determination of whether the hand is in a neutral position: a memory in communication with the processor and configured to record measurements from the sensors as measurement data: a power supply configured to supply power to the motion pod: and a communication module configured to communicate with a network to transmit the measurement data for processing.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIGS. 1A shows a range of a thumbs up position of a hand of a user.

FIGS. 1B-1H show examples of a neutral, thumbs up position of a hand of a user during various tasks.

FIG. 2 shows a classification of 3D motion data for 3 hand movements:

FIG. 3A shows a front side view of an embodiment of a pod in accordance with an aspect of the present disclosure.

FIG. 3B shows a glove and motion pod worn on a hand of a user in accordance with an embodiment of the present disclosure.

FIG. 3C shows a band and motion pod worn on a hand of a user in accordance with an embodiment of the present disclosure.

FIG. 3D shows a band and motion pod worn over a glove on a hand of a user in accordance with an embodiment of the present disclosure.

FIG. 3E is a schematic representation of a system in accordance with an aspect of the present disclosure.

FIG. 3F is a schematic representation of the sensors shown in FIG. 3A in accordance with an embodiment of the present disclosure.

FIG. 4 is a flow chart of a method in accordance with an aspect of the disclosure.

FIG. 5 depicts a high-level block diagram of a computing device suitable for use with embodiments of the system in accordance with the present principles.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Before describing embodiments of the present invention in detail, the terminology used herein is for the purpose of describing particular embodiments only and is not intended to be limiting. The invention should not necessarily be limited to specific compositions, materials, designs or equipment, as such may vary. All technical and scientific terms used herein have the usual meaning that is conventionally understood by persons skilled in the art to which embodiments of this invention pertain, unless context defines otherwise. Also, as used in this specification and the appended claims, the singular forms “a,” “an,” and “the” may include plural referents unless the context clearly dictates otherwise.

Repetitive tasks of the hand have a high-risk of hand disorders, namely carpal tunnel syndrome and wrist tendinopathy. Some risk factors include force and repetitions. Increasing the number of repetitions will increase the risk of a distal upper extremity outcome as will increasing exposure of said risk factors. Moreover, repetitive work, even at low force, can cause common hand and wrist pathologies such as carpal tunnel and wrist tendinopathy. Another risk factor is prolonged exposure. Chronic repetitive tasks can result in inflammation and other impairments that cause tissue degeneration and functional impairment. Another risk factor is awkward postures. For example, non-neutral postures increase risk of hand and wrist pathologies: working in non-neutral postures increases exertion levels and muscle force to complete movements: and intracarpal canal pressure, which is linked to carpal tunnel and wrist tendinopathy, is lowest in neutral, and increases via a parabolic relationship with the wrist: the greater the deviation, the higher the pressure. Yet another risk factor is speed. Higher angular velocity of the wrist is a predicator of musculoskeletal wrist injuries. Repetitive tasks cause tissue degeneration and adaption, but those are accelerated when the repetitive tasks occur at a faster velocity. Another risk factor is contact stress. Contact stress (pressure on the body) in isolation is not considered to be a major risk factor, but in the event that more than one risk factor is present, it is considered to be a significant contributor to risk. Other potential risk factors include vibration, psychosocial stress, age, and environment. The same risk factors affect both dominant and non-dominant hands and wrist.

Neutral positions help to limit awkward postures and reduce overall risk by keeping the body in the optimal position to provide strength, control movement, and cause the least amount of physical stress on the joint and surrounding tissues, specifics for the wrist are as follows. Thus, by identifying when a user's hand is in a non-neutral position, it may be possible to advantageously alert a user that their hand is in a non-neutral position and to thereby correct the user's hand posture. Also, it may be possible to further study why the user's hand is in a non-neutral position and to possibly improve the ergonomics of a user's work environment to eliminate or reduce the amount of time the user's hand is in a non-neutral position.

Working in a non-neutral or awkward wrist position puts stress on the tendons and tendon sheaths in the hand and wrist due to friction that is created between the hard bones and ligaments that overtime can become irritated and inflamed leading to injuries such as carpal tunnel syndrome and wrist tendinopathy. Neutral wrist postures help to reduce muscle activity which can lead to repetitive motion related injuries. Neutral wrist postures optimize force absorption as compared to non-neutral postures Neutral wrist postures reduce carpal tunnel pressure as compared to non-neutral postures.

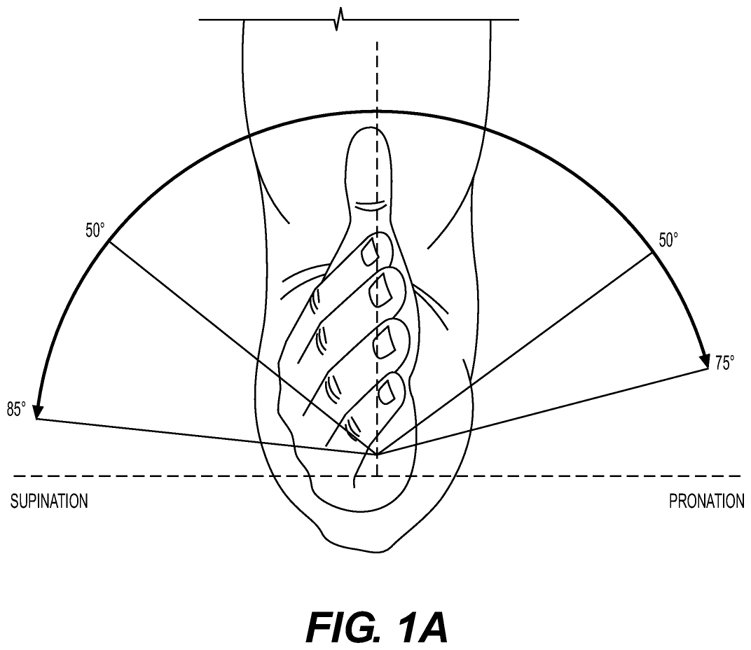

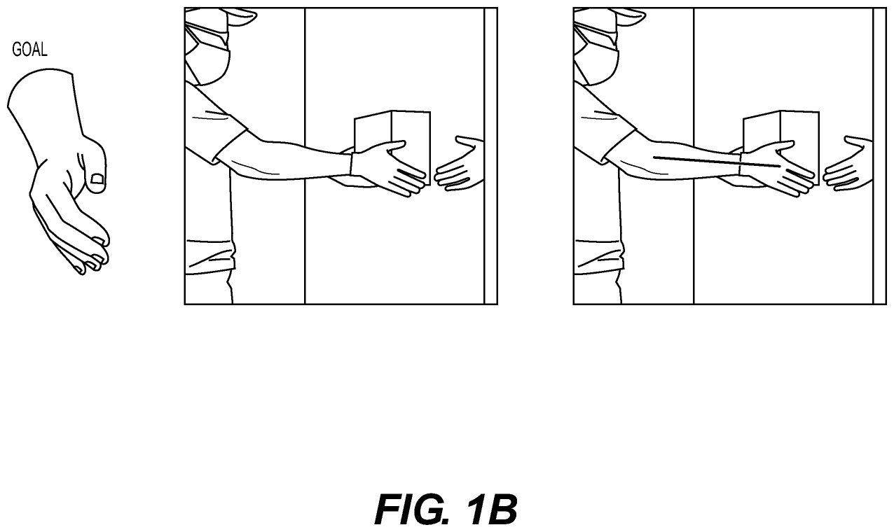

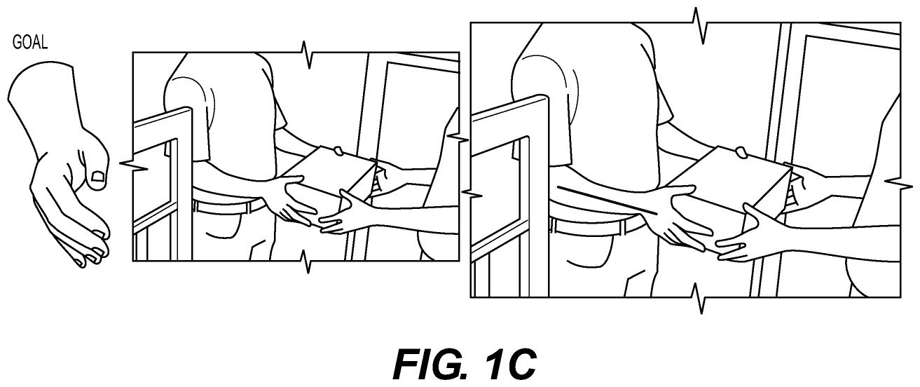

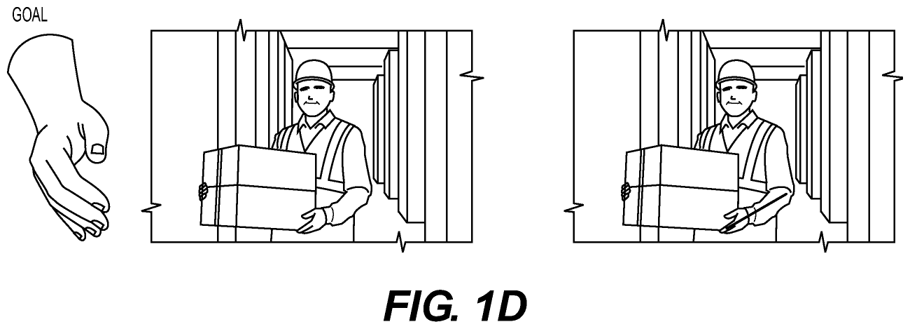

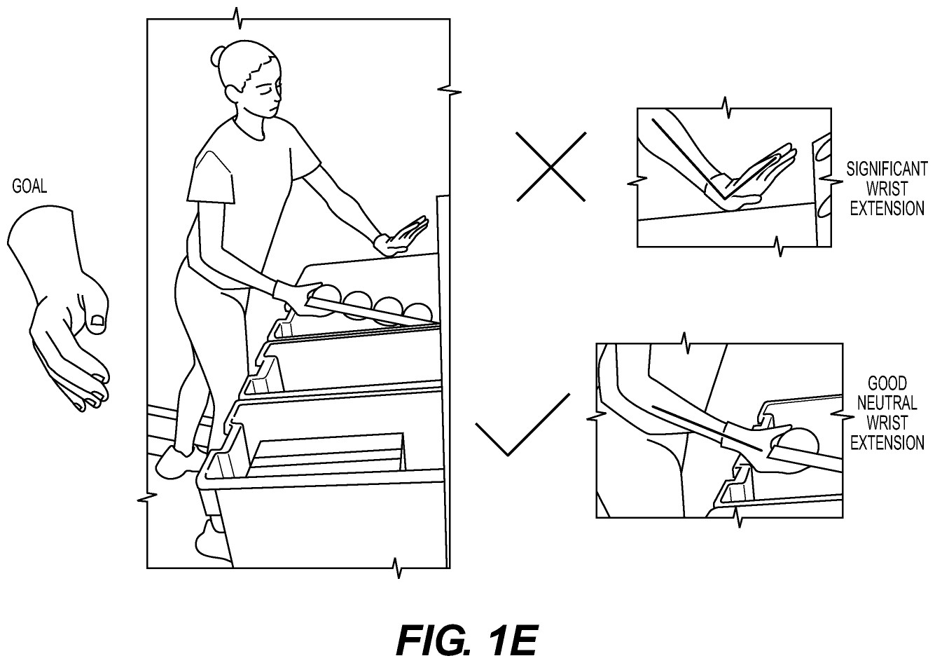

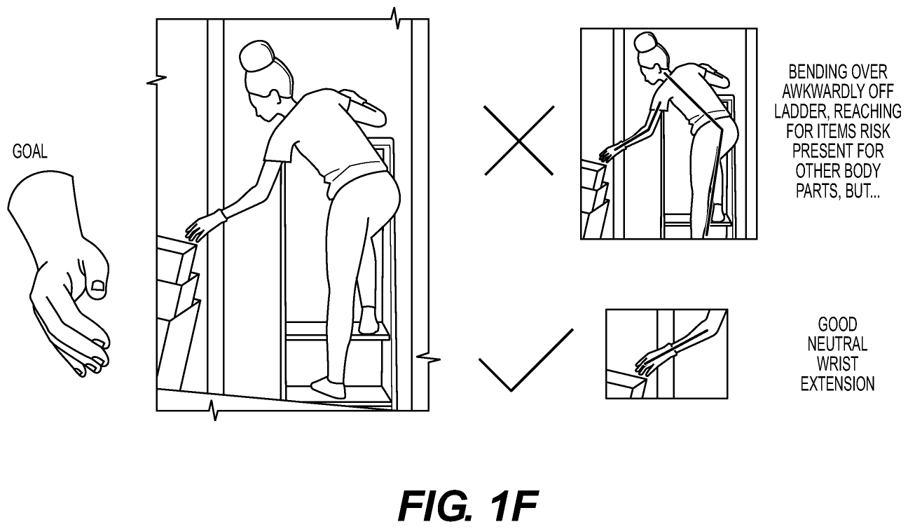

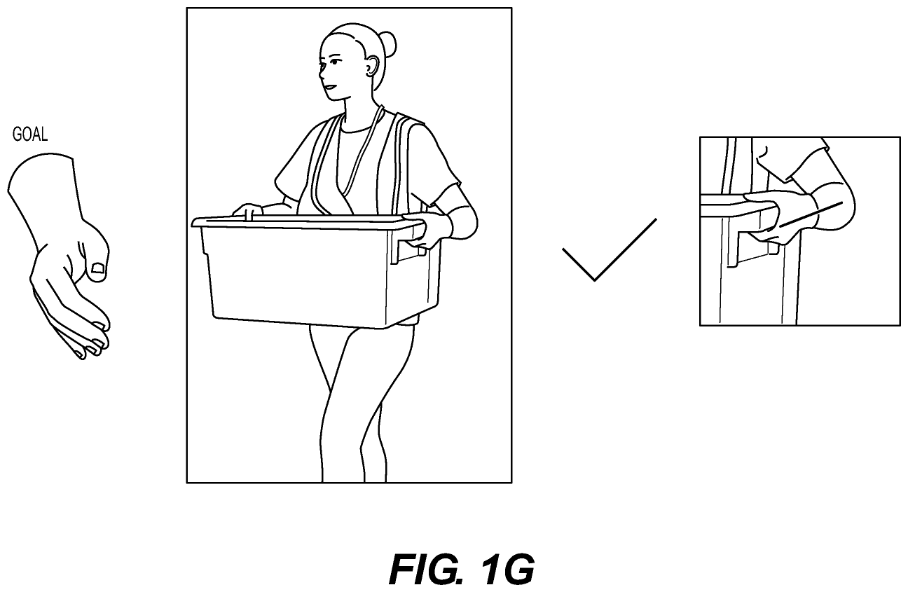

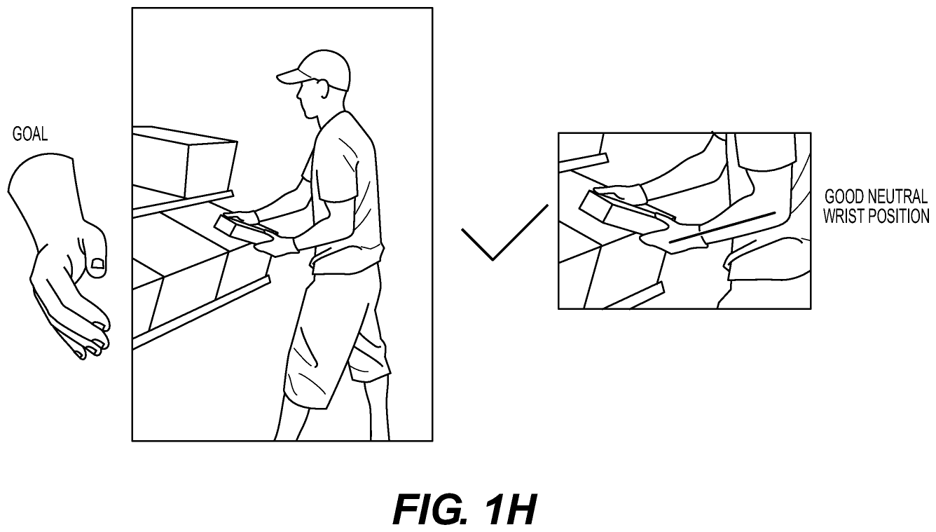

As used herein, a neutral hand and wrist position occurs when the wrist is in the same plane as the forearm and a straight line can be drawn from the elbow to the fingers: this is referred to herein also as a ‘thumb up position.’ The thumbs up position is illustrated in Fig. 1A. Also, the neutral wrist position can be found by dangling the arms at one's side, the position your hands are in when you hold a steering wheel at the 10 o'clock and 2 o'clock positions, or when offering someone a handshake. More specifically, in anatomical terms, a neutral position is considered to be within a thumbs up zone (also referred to herein as a “neutral zone”) defined by a range of 0 to 30% of maximum range of motion, 0-9 of Flexion, 0-9 of Extension, 0-4.5 of Ulnar Deviation, 0-3 of Radial Deviation, 0-10.5 of Supination, 0-12.75 Pronation. Also, non-neutral hand and wrist ranges (based on conservative Max ROM) (Moderate 15-50% of max ROM, Significant 50-75% of max ROM, End Range 75-100% of max ROM) are: Flexion: (Moderate 9-30), (Significant 30-45), (End Range 45°+), Extension: (Moderate 9°-30°), (Significant 30°-45°), (End Range 45°+), Ulnar Deviation: (Moderate 4.5°-15°), (Significant 15°-22.5°), (End Range 22.5°+), Radial Deviation: (Moderate 3°-10°), (Significant 10°-15°), (End Range 15°+), Supination: (Moderate 10.5°-35°), (Significant 35°-52.5°), (End Range 52.5°+), Pronation: (Moderate 12.8°-42.5°), (Significant 42.5°-63.8°), (End Range 63.8°+). Examples of neutral hand and wrist positions for workers performing various repetitive tasks are illustrated in FIGS. 1B-1H.

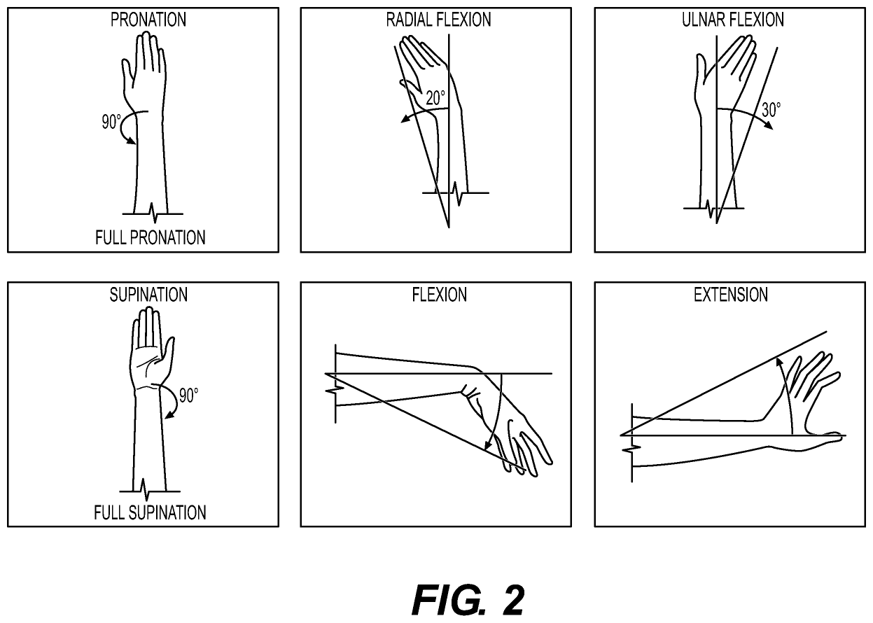

FIG. 2 shows three-dimensional hand motion classification into ergonomic movements. For example, the illustration of flexion/extension movements are like those used in painting with a brush. Also, the illustration of pronation/supination movements are like those used in operating a doorknob. The illustration of radial/ulnar flexion movements are like those used for waxing a surface. Each hand movement may be further classified by speed of motion as either “fast” or “slow” to create six unique hand movements. Faster hand or wrist motions are more likely to lead to repetitive strain injury, whereas slower hand or wrist motions are less likely to lead to such injuries.

In embodiments, systems and methods in accordance with the present disclosure track hand or wrist movements and positions to identify when a hand of a user (also referred to interchangeably herein as an “active user”) is in a neutral position and non-neutral position. Such measurements may be used advantageously to mitigate risk of injuries, including injuries caused by repetitive motions, and can be useful for enhancing productivity and ergonomics.

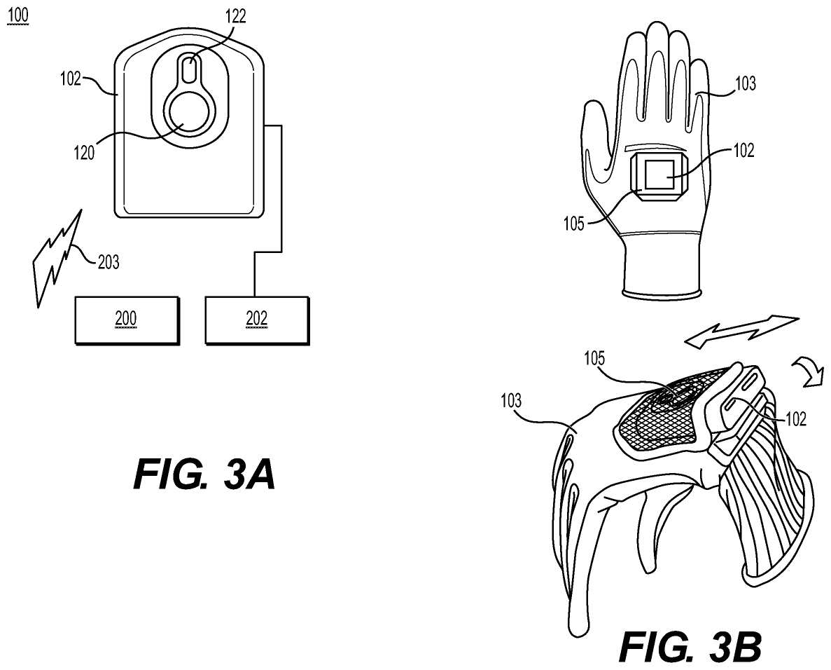

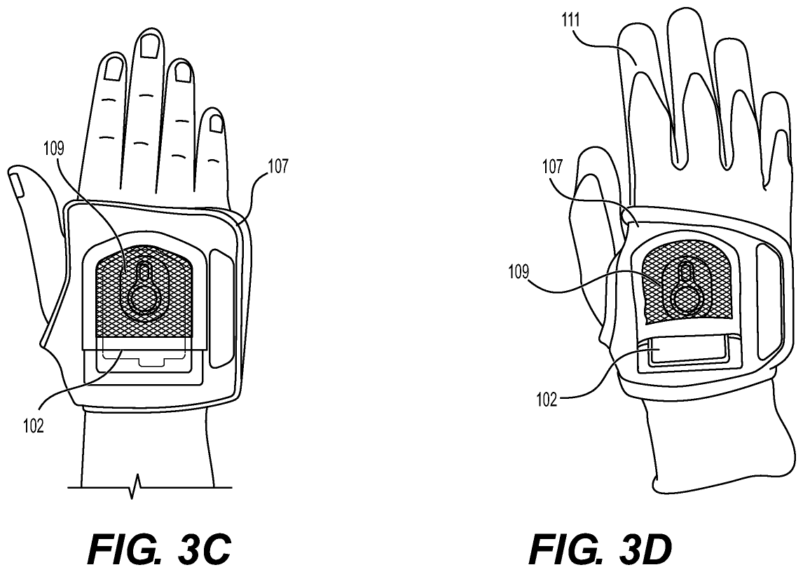

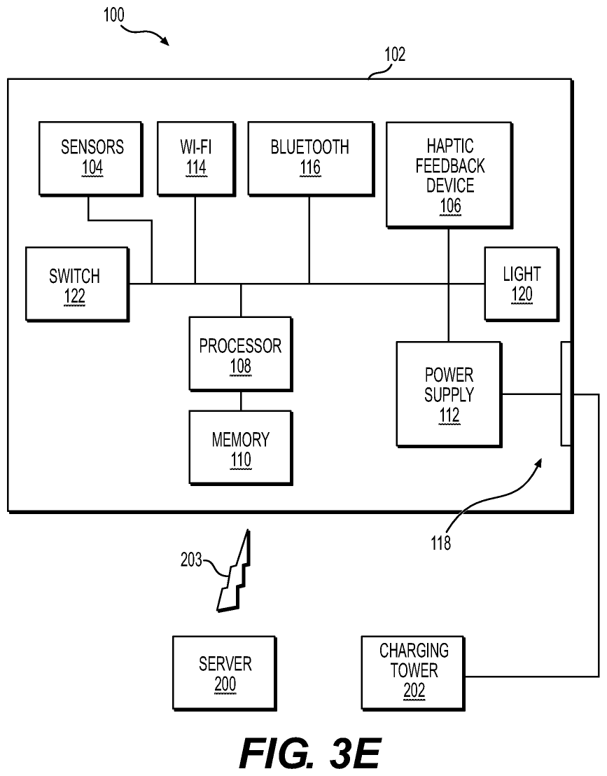

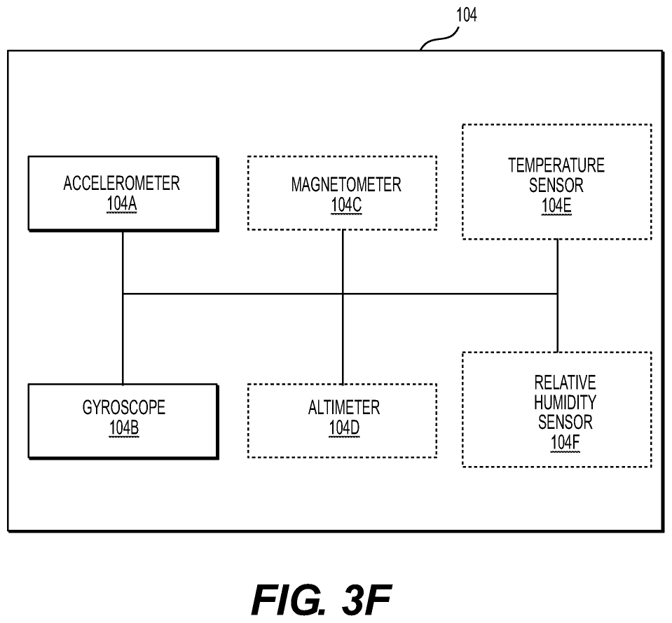

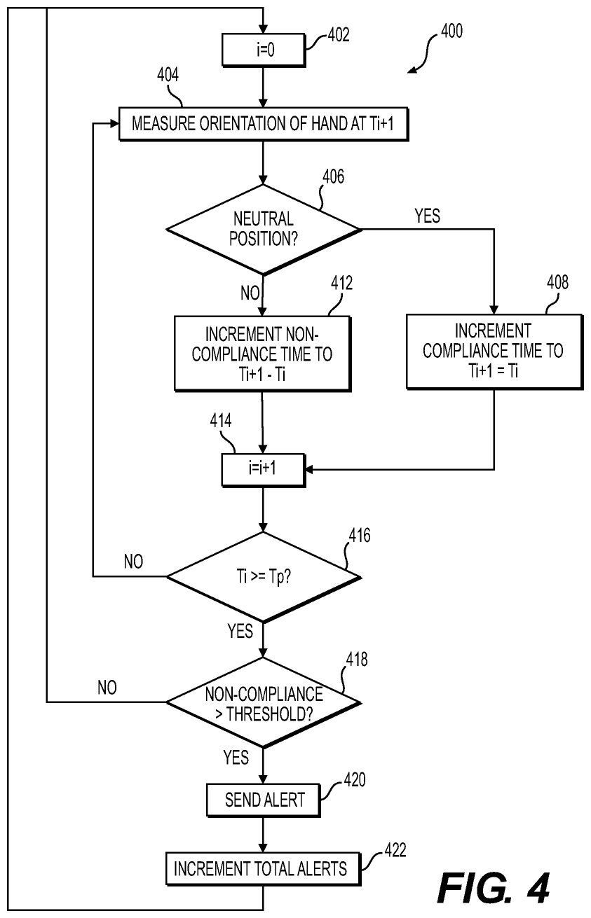

FIG. 3A shows an embodiment of a wearable system 100 configured for measuring at least one of hand and wrist posture and movement of a user. In embodiments, the system 100 includes a wearable pod 102 a server 200 (also referred to herein more generally as a “data processing system”), and a charging tower 202. The pod 102, server 200, and charging tower 202 may be configured to be communicatively coupled, such as by wire or wireless connection. For example, the pod 102 may be connected to the server 200 through network 203, which may be any type of communication network. The pod 102 is configured to monitor hand or wrist motion and movement of a user in three dimensions as a time series or recorded data (otherwise referred to herein as measurement data). Additional details of the system 100 are discussed in greater detail below.

In some embodiments the pod 102 may be worn, for example, on a wrist or a hand of a user. For example, as shown in FIG. 3B, the pod 102 may be physically connected (e.g., removably connected) to a back side (opposite a palmar side) of a glove 103 worn on a hand of a user. As shown in FIG. 3B, the glove 103 may have a pocket 105 or pouch to house the pod 102 on the back of the glove 103.

FIG. 3C shows another embodiment where the pod 102 is removably connected to a strap 107 or band worn on a bare hand of a user with the pod 102 positioned on a back side of the hand in a pocket 109 of the strap 107. FIG. 3D shows the strap 107 of FIG. 3C worn over a glove 111 worn on a hand of the user. The gloves 103, 111 and band 107 may be personal protective equipment (PPE). While the pod 102 is shown being removably connected to the glove 103 and the band 107, in some embodiments, the pod 102 and the glove 103 and the band 107 are integrally connected. In some embodiments, the pod 102 may be attached directly to the skin (e.g., of the back of a hand or on the wrist) of a user with an adhesive. For example, in some embodiments a double-sided adhesive tape may be used to attach the pod 102 to the skin.

FIG. 3E shows an embodiment of the system 100 with a schematic view of an embodiment of the pod 102. The pod 102 comprises a processor 108, a memory 110, and support circuits (sensors 104, haptic feedback module 106, power supply 112, Wi-Fi communication module 114, Bluetooth communication module 116, light 120, and switch 122). The processor 108 may comprise one or more commercially available microprocessors or microcontrollers that facilitate data processing and storage. Optionally, the pod 102 may have other or additional types of data communication modules, such as cellular telephony module, NFC, RFID, etc. The various support circuits facilitate the operation of the processor 108 and may include one or more clock circuits, power supplies, cache, input/output circuits, and the like. The memory 110 comprises at least one of Read Only Memory (ROM), Random Access Memory (RAM), disk drive storage, optical storage, removable storage and/or the like. In some embodiments, the memory 110 comprises an operating system and a haptic feedback training program for execution by the processor 108 to carry out at least a portion of a haptic feedback training module, in accordance with an aspect of the disclosure. Also, the memory 110 is configured to store measurement data obtained using the sensors 104.

In some embodiments, such as that shown in FIG. 3F, the sensors 104 include an accelerometer 104a and a gyroscope 104b. In some embodiments, such as that shown in FIG. 3F, the sensors 104 may also include a magnetometer 104c. In embodiments, the accelerometer 104a is configured to measure the speed of movement of the pod 102, and, thus, the speed of movement of the hand of a user on which the pod 102 is worn. In embodiments, the gyroscope 104b is configured to measure an angular deflection of the pod 102 and, thus, the angle of the hand of the user (e.g., in pronation or supination) on which the pod 102 is worn. Also, in embodiments, the magnetometer 104c is configured to measure a displacement and position of the pod 102 and, thus, of the user's hand when the pod is worn on the hand or wrist of a user.

Optionally, in some embodiments, such as that shown in FIG. 3F, the sensors 104 may also include at least one of an altimeter 104d, a temperature sensor 104e, or a relative humidity sensor 104f. In embodiments, the altimeter 104d is configured to measure a vertical height of the pod 102 and, thus, the height of the hand of a user when the pod 102 is worn on a hand or wrist of a user. In embodiments, the temperature sensor 104e is configured to measure the ambient temperature near the pod 102 and, thus, near the hand of the user when the pod 102 is worn on a hand of the user. In embodiments, the relative humidity sensor 104f is configured to measure the ambient relative humidity near the pod 102 and, thus, near the hand of the user when the pod 102 is worn on a hand of the user. In some embodiments, temperature and relative humidity may be monitored by the system 100 to monitor the environmental conditions of the user wearing the pod 102. For example, if the user is a worker operating in extreme temperature and/or humidity environments, such as a freezer or oven, the amount of exposure of the worker to extreme temperatures may be measured and recorded by the system 100.

In some embodiments, the haptic feedback device 106 is configured to provide haptic sensory feedback to the wrist or hand of a user wearing the pod 102 to alert the user that they are out of compliance with a haptic training program limit for neutral hand and wrist posture. In some embodiments, the sensory feedback may include at least one of haptic feedback (buzzing), visual feedback (e.g., lights), or audio feedback (e.g., sounds or buzzing). In some embodiments, combinations of haptic, visual, and/or audio feedback is provided to the user to ensure the feedback is experienced by the user in all environmental operating conditions so corrective action may be taken by the user. In some embodiments, haptic feedback is at least provided and may be supplemented by at least one of visual and/or audio feedback.

In some embodiments, the haptic feedback device 106 includes a vibration motor configured to vibrate the pod 102 and cause haptic feedback via a buzz or vibration to be transmitted to the wrist or hand of the user wearing the pod 102. In embodiments provided herein, the haptic feedback (also referred to as tactile feedback) may use vibration patterns, waveforms, forces, or motion to convey information to a user or operator. Exemplary methods of using the pod 102 to provide haptic feedback to a user are described in greater detail herein. In some embodiments, the haptic feedback device 106 includes lights to provide visual feedback and/or a speaker or buzzer to provide audio feedback.

In some embodiments, the wireless communication module 114 is configured to wirelessly communicate (e.g., unidirectionally or bi-directionally) with the remote server 200 and/or the charging tower 202. For example, in some embodiments the wireless communication module 114 is configured to transfer the measurement data to the remote server 200. Also, in some embodiments, the wireless communication module 114 may be used to download over-the-air updates to the processor-executable program (e.g., firmware) stored in memory 110 as needed to the pod 102.

In some embodiments, the remote server 200 is configured to process and perform an analysis of the measurement data received from the pod 102. In some embodiments, the server 200 uses a remote cloud service to process and/or perform the analysis of the measurement data. For example, the remote server 200 may employ an artificial intelligence (AI) system to provide user-specific feedback and training recommendations to the user based on the measurement data from the pod 102 worn by the user. Also, in some embodiments, the user-specific feedback and training recommendations are based additionally on measurement data obtained from pods 102 worn by other uses who may be peers of the user and grouped with the user in some manner related to ergonomics (e.g., other uses performing similar hand motions or tasks as the user). A comparison to other uses allows for benchmarking each user against their peers. Also, in some embodiments, parameters used by the program executed by the processor 108 may be modified based upon the analysis of the remote server 200. Further details of the use of the measurement data for user recommendations and training are provided herein.

The Bluetooth module 116 may be configured to communicate with other Bluetooth enabled devices, such as other pods 102. In embodiments, such communication may be enabled to monitor the proximity between pods, and, thus, between users wearing the pods (e.g., social distancing). In another embodiment, Bluetooth communication may be used to locate a missing pod 102. In yet another embodiment, a single user may wear multiple pods configured to communicate with one another using Bluetooth communication. Such multiple pods may be used to track relative positions between different parts of the body of the user, such as hand and shoulder.

In some embodiments, the light 120 may be configured to provide feedback to the user wearing the pod 102. In embodiments, the light 120 is configured to illuminate in different colors. For example, in embodiments, the light 120 may be configured to illuminate blue when the pod 102 is powered on, purple when the pod 102 is charging and not connected to the server 200, green when the pod 102 is being charged and connected to the server while transferring data, white when the pod 102 is charging and all data has been transferred or if not being charged, the memory 110 of the pod 102 is full, red if the power supply is less than 10% of capacity or to indicate a failure of the pod 102, flashing purple and white if the pod 102 is connected to a charger, such as charging tower 202.

In some embodiments, the switch 122 may be configured to turn the pod 102 on and off. In some embodiments, the switch 122 may be configured to interact with a user, such as to acknowledge sensory feedback being generated by the haptic feedback device 106. For example, the switch 122 may be a button that can be depressed by a user to acknowledge and terminate the sensory feedback. In some embodiments, the switch 122 and the light 120 may be integrated into a lighted pushbutton.

The electric power supply 112 is configured to supply power to the pod 102. In the schematic representation shown in FIG. 3A, the power supply 112 is part of the pod 102. However, in some embodiments, the power supply 112 may be external to the pod 102 and be connected via a wired or wireless connection (i.e., inductive coupling). The electric power supply 112 may be an AC or DC power supply. In some embodiments, the power supply 112 is a battery and is connected to a wired interface 118, which may include electrical contacts configured for contacting a battery charger (not shown) for charging the battery. In some embodiments, the battery charger may be configured to charge a pod individually or charge a plurality of pods at the same time. For example, in some embodiments, the charging tower 202 includes a battery charger configured to charge at least one pod 102 while the pod 102 is connected to the charging tower. In some embodiments, the charging tower 202 may be configured to independently charge one or more pods 102 connected to the charging tower. In some embodiments, the pod 102 is configured to communicate with the server 200 via wi-fi communication when the pod 102 is being charged. In some embodiments, to conserve power, the pod 102 is configured to not communicate with the server 200 when the pod 102 is not being charged. In yet other embodiments, the pod 102 may be configured to communicate in real time or variously (e.g., periodically) with the server 200.

In embodiments, the charging tower 202 includes one or more charging receptables, docks, or connectors to electrically connect to the wired interface 118 of respective pods 102. In some embodiments, the charging tower 202 houses one or more pods 102 when pods 102 are not being worn by a user. In some embodiments, a user may interact with the charging tower 202 when checking out a pod 102 for use or when returning the pod 102 when the user is finished using the pod 102.

The charging tower 202 may include or be connected to an allocation controller to control the check out and check in of the pods 102. More specifically, in some embodiments, the allocation controller is configured to control the assignment and/or reassignment of each pod 102 to a user. The allocation controller may include a terminal and a user interface for interaction with a user.

In some embodiments, the allocation controller is configured to perform a static allocation of one pod 102 to one user, whereby each pod 102 is statically (continuously) assigned to the same user in one-to-one correspondence. In some embodiments, the allocation controller is configured to perform a dynamic allocation of pods 102 to users so that each pod 102 can be assigned to any user based on various factors, such as state of charge of the pod 102, state of memory transfer of the pod 102, and the anticipated usage of the pod 102 by the user (i.e., will the pod 102 be able to operate for the entire shift of the user).

In a dynamic allocation arrangement, in some embodiments, a user interacts with the user interface (e.g., a GUI) of the terminal of the allocation controller for entry of user input to check out a pod 102 from the charging tower 202. Before a user is issued a pod 102 from the charging tower 202 to use (such as at the beginning of a work shift), a user may input their user ID or other identifying credentials associated with the user, such as job site and task information, which can be used by the allocation controller to select a pod 102 for the user to use during the work shift. Such input may be done by scanning a barcode of a user ID badge or via other means, such as a keypad, fingerprint scanner, retinal scan, voice, or other biometric input. As noted above, in some embodiments, the allocation controller may assign a pod 102 to a user with sufficient battery charge and memory storage to last for the duration of the user task (i.e., the user shift).

When the user is finished using the pod 102, the user can return the pod 102 to the charging tower 202 to check the pod 102 back in to charge the pod 102 and permit the transfer of collected measurement data from the pod 102 to the server 200. In some embodiments, docking the pod 102 in the charging tower 202 automatically triggers a download of measurement data from the pod 102 to the server 200 for automated data analysis and reporting (e.g., to the user or managers).

In some embodiments, the server 202 is configured to communicate with the pod 102 to receive measurement data from the pod 102 and/or send program updates to the pod 102. In some embodiments, the server 202 may be used for handling, storage, and computation of measurement data received from the pod 102. The server 202 may also utilize inbound cloud service(s) to perform one or more of handling, storage, or computation of measurement data received from the pod 102. For example, in some embodiments, an artificial intelligence (AI) engine running on the server and/or in the cloud may be used to identify and classify unique ergonomic movements and orientation using the measurement data. In some embodiments, a mobile application (“app”) running on a computing device (e.g., a mobile computer or cell phone separate from the pod 102) associated with the user may be used to display or otherwise report analyzed data and insights directly to the user. Also, in some embodiments, the mobile app can be used by a manager or supervisor to review reports and analyses generated from the measurement data. More specifically, in at least one embodiment, the mobile app may be configured as a web dashboard to administer the system 100 and provide data insights to group users as well as a customized training platform (Programs, Modules, Unit content, Dashboard, closed-loop controls).

As noted above, in some embodiments, the pod 102 may be mounted on the back of the wearers hand or wrist and configured to track hand and wrist posture of a user when the pod 102 is worn on the hand or wrist of the user. For example, in embodiments, the pod 102 uses the plurality of sensors 104 to measure, in real time, orientation and movement of a wrist or hand of the user and determine whether the measurements are within the thumbs up zone. In some embodiments, the processor 108 periodically tracks the time and thumbs up zone status of the hand and stores the information in memory 110. The processor 108 is configured to determine whether the hand is in a neutral position based on the movement and orientation measurements of the sensors 104. For example, as discussed above, in some embodiments, if the measurements from the sensors 104 indicate that the hand of the user is in a thumbs up zone, then the processor 108 determines that the hand is in a neutral position, but if the measurements from the sensors 104 indicate that the hand of the user is not in the thumbs up zone, then the processor 108 determines that the hand of the user is in a non-neutral position.

For example, in some embodiments, the measurements from the accelerometer 104a and the magnetometer 104c may be used to determine/calculate if the hand is in a thumbs up zone, and then uses the gyroscope 104b to measure an angle of deviation in supination or pronation motions over time to determine whether the hand moves in or out of the thumbs up zone. In some embodiments, the sensors 104 may also be configured to measure an angular range of flexion, extension, ulnar deviation, and radial deviation.

In some embodiments, the processor 108 is configured to determine an amount of time that the hand is in the neutral position and/or non-neutral position. In some embodiments, if the processor 108 determines that the hand is in a non-neutral position for a certain percentage of a measurement time window, the processor 108 communicates with the haptic feedback device 106 by sending a haptic feedback signal to the haptic feedback device 106 to activate the haptic feedback device 106. More specifically, in some embodiments, the processor 108 is configured to calculate a non-compliance percentage equal to a percentage of time in a certain measurement time window that the user's hand is outside of the neutral position and compare the non-compliance percentage to a certain non-compliance limit or threshold, which may be adjusted automatically or manually over time, as discussed in greater detail herein below. In some embodiments, the processor 108 is configured to send the haptic feedback signal to the haptic feedback device 106 if the calculated non-compliance percentage is at or above the non-compliance limit. This is referred to herein as a THUMBS-UP feedback feature.

Also, in some embodiments, the system 100 may record a metric measuring time in compliance (i.e., hand in thumbs up position) per measurement time window or period. The duration of the measurement time window or period, the % of time “out of compliance,” and the degree of difference from neutral can all be adjusted. For example, the memory 110 may store the duration of the time period, the % of time “out of compliance,” and the degree of difference from neutral as values which may be used by the program stored in the memory 110 during execution of the program by the processor 108. Those parameter values may be overwritten or otherwise changed over time, as discussed in more detail below. For example, in some embodiments, three variables may be set: (i) the range of motion or degree of difference: (e.g., +/−45 degrees from neutral); (ii) the measurement time window or period to monitor (e.g., starting at 2 minutes or between 1-5 minutes): and (iii) the % of time out of neutral (e.g., starting at 65% or between 50-75%). Thus, for example, if a user wearing the pod 102 is in a non-neutral position (i.e., out of compliance) for more than 65% of the time, the pod 102 generates a haptic feedback alert or notification (e.g., a vibration) using the haptic feedback device 106, thereby alerting or notifying the user to modify their hand posture to use the neutral position more often. In embodiments, a counter may reset after each measurement time window or period.

In some embodiments, the total number of haptic feedback alerts or notifications that are generated by the haptic feedback device 106 during a certain time period or window (e.g., amount each day or work shift) are tracked as a metric for training purposes. In addition, in some embodiments, the system 100 may calculate the total amount of time spent in neutral position that day or shift (for example, user spent 25% of his time in “Thumbs up” position. This allows the system 100 and users to identify activity trends and areas of improvement for training purposes. The total number of haptic feedback alerts and total amount of time spent in neutral position can be calculated by the processor 108 or by the server 200.

FIG. 4 shows an embodiment of a closed-loop haptic feedback training method 400 in accordance with the present disclosure. At 402 a measurement counter is initialized to zero and a sequence of measurements commence over a certain period of time, such as 2 minutes. At 404, the movement and orientation of the wrist or hand of the user is measured using the sensors 104, as described above. At 406 the system 100 determines from the measurement data whether the hand of the user is in a neutral position. For example, the neutral position may be defined as a range of measurements defining the thumbs up zone so that measurements within the thumbs up zone will be determined to be in the neutral position and measurements outside of the thumbs up zone will be determined to not be in the neutral position. In one example, the thumbs up zone may include +/−30 degrees pronation/supination from the center position shown in Fig. 1A. However, it will be understood that the range of degrees defining the thumbs up zone can be adjusted to be anything, such as, for example, +/−45 degrees pronation/supination from the center position shown in FIG. 1A.

If the hand of the user is determined to be in a neutral position (YES at 406), then the total compliance time is incremented at 408 and the counter is incremented at 414. The increment may be, for example, 10 seconds. Otherwise, if the user hand is not in a neutral position (NO at 406), then the non-compliance time is incremented at 412 and the counter is incremented at 414. At 416 a determination is made as to whether the measurement window has expired. If the measurement window has not expired (NO at 416), the orientation and movement measurement of the writs or hand is repeated at 404. If the measurement window has expired (YES at 416), then a determination is made about whether a total non-compliance time percentage is greater than a threshold percentage. For example, the threshold percentage is a percentage of time of the measurement window where the user is not in a neutral position. In one example, the threshold percentage is 65%. If the total non-compliance time percentage is greater than the threshold (YES at 418), then the haptic feedback device 106 generates a haptic feedback alert at 420 and the total number of haptic feedback alerts is incremented at 422, after which the counter and the measurement time window are reset to zero at 402. Thus, in the example mentioned above, if a user is not in a neutral position more than 65% of the 2-minute measurement window, the pod 102 will generate a haptic feedback alert. Otherwise, if the total non-compliance time percentage is less than the threshold (NO at 418), the counter and the measurement time window are reset to zero at 402 and the method repeats 400.

In some embodiments, the method 400 ends when the user returns the assigned pod 102 they are wearing to the charger, such as the charging tower 202, whereupon measurement data recorded by the pod 102 can be downloaded or otherwise transferred to the server 200 for analysis and reporting. Alternatively, or in addition to the foregoing embodiments, the data recorded by the pod 102 may be periodically transmitted to the server 200 to avoid waiting to transmit the data until the user returns the assigned pod 102 to the charging tower 202. Alternatively, in some embodiments, the data recorded may be transmitted continuously to the server 200. The ability of the pod 102 to transmit data periodically or continuously may be based on at least one of the availability of power stored in the pod 102, capacity of the memory 110, or the predicted power usage of the pod 102 required for recording and transmitting data.

In some embodiments, the system 100 may be configured to operate in an open loop mode, wherein the pod 102 merely records measurement data and does not provide sensory feedback to the user wearing the pod 102. In such an embodiment, any reports generated from the measurement data may be used by managers to track progress of user compliance with ergonomic training.

In some embodiments, the system 100 may be configured to operate in a closed loop mode, wherein the pod 102 also generates sensory feedback as described above to alert a user wearing the pod 102 of not being in a neutral hand or wrist posture for a certain percentage of time. Moreover, in other embodiments, the system 100 may also be configured in a closed loop mode so that the analysis performed by the server 200 and/or cloud services are used as additional feedback to modify the method 400 to facilitate and customize user training.

For example, in some embodiments, an analysis of the total number of alerts sent to a user during a working shift or other time period may be used to determine whether to adjust one or more parameters used in the method 400, such as the duration of the measurement window, the non-compliance threshold, and the measurement ranges used to define the thumbs up zone. For example, a low number of alerts may indicate that the user has mastered the current training requirements and is ready for more stringent training requirements. For example, over time (e.g., weeks or months) the range of motion and orientation defining the thumbs up zone may be reduced by a certain percentage, thereby limiting the range of motion and orientation of the user's hand and wrist that will be considered to be in the neutral position. Thus, the analysis of the measurement data recorded by the pod 102 can provide closed loop feedback to modify the method 400 to gradually train the user to move and orient their hands and wrist in a neutral position defined by a narrower range of motion. More specifically, in some embodiments, the program and/or parameters stored in the memory 110 of the pod 102 for carrying out the method 400 may be periodically modified or updated as a result of commands issued manually (e.g., by a manager or supervisor reviewing the measurement data) or automatically (e.g., by using a training algorithm used by the server 200).

The systems and methods described herein are useful to train users to reach individual and cohort (e.g., group) level training goals through an analysis of individual and cohort-level measurement data. In some embodiments, training may be performed at a group level or an individual level at various stages of a training program. Process-specific training modules may be developed by analyzing measurement data obtained from a group of users wearing pods 102 who perform specific types of repeatable processes. In some embodiments, the measurement data of the group of users may be anonymized. Best practices can be obtained from an analysis of such process-specific measurement data. Artificial intelligence (AI) may assist in identifying best practices. For example, the measurement data of the cohort can be analyzed to define an optimal sequence of steps to complete the processes while using the pod 102 that maximizes compliance with ergonomic training goals. Also, the cohort measurement data may be mined or otherwise analyzed to identify trends in best practices over time. For example, it would be expected that best practices will improve and then plateau over time until engineering controls are put in place for the group to further improve best practices. Thus, tracking the best practice trends may aid managers in determining when to deploy new engineering controls to drive ergonomic improvements.

At the user-level, training can be individualized based upon the measurement data recorded by the pod 102. As discussed above, sensory feedback to the wearer of the pod 102 will alert the user to a non-compliance with training goals. The measurement data may be analyzed to provide the user with their individual haptic violation rate or non-compliance rate. The haptic violation rate may be calculated as the number of haptic feedback alerts generated by the pod 102 per period of time (e.g., over each measurement period). The analysis of the user measurement data may also be reported in the afore-mentioned mobile app to track user metrics, such as hours that the pod 102 is worn, movement rates, haptic violation rate, and movement mix. Also, the measurement data can be analyzed to provide the user with individual recommendations for actions to take to improve the metrics to achieve group best practices, as discussed more fully below.

Also, individual user training may be based on cohort-level data analysis. In some embodiments, users who wear a pod 102 may be assigned to a cohort of users who wear a pod 102 for a specific process. As discussed above, in some embodiments, the measurement data obtained for the cohort may be analyzed to determine benchmark or best practices for various metrics associated with the specific process, including: haptic violation rate, movement rates, and movement mix. Also, risky practice metrics can be generated based on the measurement data for the assigned cohort. Such risky practice metrics include haptic violation rate, movement rates, and movement mix that are used to define upper limit thresholds for each metric to identify risky practices based on all active users in the cohort for a specific process.

Also, hand or wrist movement metrics can be derived from the cohort measurement data. Such hand or wrist movement metrics include movement rates and movement mix that assess hand/wrist safety for each active user against best practice or the benchmark. Also, cohort risk stratification metrics can be derived from the cohort measurement data. Such metrics include ranking cohort for defined period (day, week) based on a leading indicator (haptic violation rate, movement rate, etc.) to identify top quartile (25%) and top decile (10%).

The aforementioned cohort-level metrics can be used for feedback and control of the training program for individual users. For example, in some embodiments, exceeding an upper limit threshold (Risky Practice) for the haptic violation rate over a specific time period (e.g., over a work shift) may trigger a notification to the user that they are required to repeat completion of process-specific haptic program training module for the user. Also, in some embodiments, exceeding the upper limit threshold(s) (Risky Practice) for movement rate(s) over a specific time period (e.g., over a work shift) may trigger a notification to the user that they are required to repeat completion of process-specific best practice training module. Also, in some embodiments, users who are in either the top decile or top quartile based on risk stratification may be notified that they are assigned to complete a process-specific injury prevention program training module. Also, in some embodiments, users can use the afore-mentioned mobile app to track hours the pod 102 is worn, and the user's haptic violation rate and movement rate over time. In addition, the mobile app may provide feedback to the user with user-specific, tailored recommendations for the user to close any gaps between the user's metrics and the best practices based on cohort-level metrics.

Various administrative procedures and controls may be put in place to support the user training. For example, in some embodiments, a 12-month refresher training program may be required whereby users who complete an initial training program for a specific process are notified to complete a refresher training program annually. During the refresher training program, a wearable pod 102 is worn by the user for a certain period of time (e.g., 2-4 weeks) to provide closed-loop control and feedback, in addition to training module(s), so users can compare their metrics during the refresher training program to latest best practices. In some embodiments, administrative controls may be implemented for users who complete injury prevention program training module(s) and who remain at highest risk (e.g., top decile or quartile) for injury are identified so that a safety team can take additional actions such as 1-on-1 coaching of the user, job rotation of the user to limit time on process, etc.

The systems and methods described herein can be utilized to mitigate occupational hazards and can be directly applied to maintaining a neutral wrist and hand posture. The systems and methods described herein provide at least one of a visual (e.g., lights), audio (e.g., sounds or buzzing), or tactile indication (e.g., haptic feedback due to non-neutral posture or repetitive motion) of potential ergonomic hazards allowing for quicker and more seamless mitigation to occur. In some embodiments, combinations of haptic, visual and/or audio feedback is provided to the user to ensure the feedback is experienced by the user in all environmental operating conditions so corrective action may be taken by the user. Moreover, utilization of feasible control strategies can assist in reduction of risk in two primary ways: controlling the hazard and working to control the exposure to the hazard.

Controlling the hazard may involve physical changes to the work environment to limit workplace hazards, such as wearing the pod 102. For example, hazards may be controlled by eliminating the hazard or substituting a reduced risk for the hazard, such as, for example, working to remove the need for workers to deviate their wrist from the neutral posture. Hazards may be eliminated or reduced by reducing hazards at their source through the design of tools, equipment workstations, and/or machines to relieve physical stress. In the case of repetitive hand injuries, one example of engineering controls is to utilize tools and workstation equipment that promotes neutral wrist posture. Additionally, the workplace may be modified to reduce worker exposure to the hazard.

In addition to the foregoing controls, management controls may be employed to establish workplace rules/guidelines/policies to minimize exposure to hazards as well as processes and procedures to train/inform/educate the workforce of successful ways to limit exposure. An example of a management control is the use of the measurement data recorded by the pod 102 for feedback to enhance training of the user, as discussed hereinabove. Another example is a microbreak recommendation and task rotation schedule. Also, behavioral controls may be used to control hazards. For example, job coaching and specific training modules designed around education of safe work practices, improving neutral posture, and maximize workplace body mechanics may be utilized.

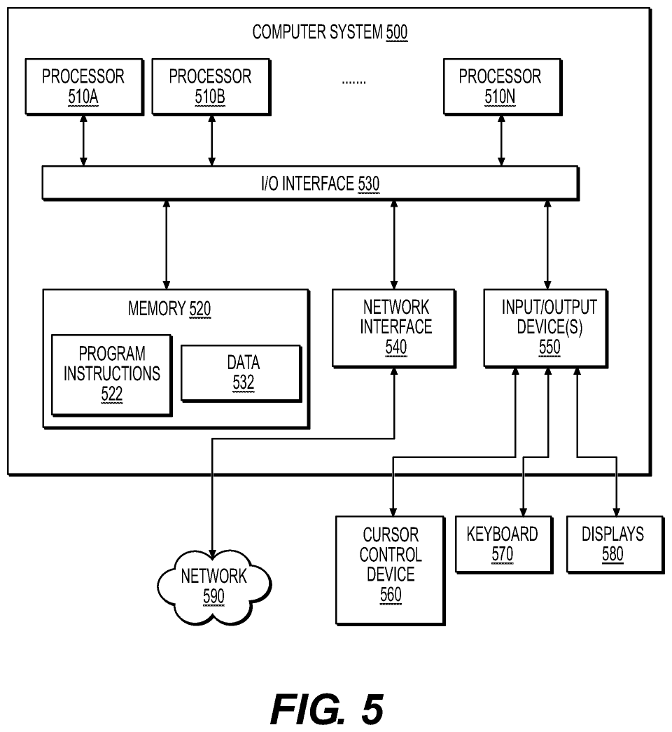

FIG. 5 depicts a computer system 500 that can be utilized in various embodiments of the invention to implement one or more of the pod 102, server 200, and charging tower 202, according to one or more embodiments.

Various embodiments of method and system may be executed on one or more computer systems, which may interact with various other devices. One such computer system is computer system 500 illustrated by FIG. 5, which may in various embodiments implement any of the elements or functionality illustrated in FIGS. 1A-4. In various embodiments, computer system 500 may be configured to implement methods described above. The computer system 500 may be used to implement any other system, device, element, functionality, or method of the above-described embodiments. In the illustrated embodiments, computer system 500 may be configured to implement the method 400 as processor-executable executable program instructions 522 (e.g., program instructions executable by processor(s) 510) in various embodiments.

In the illustrated embodiment, computer system 500 includes one or more processors 510a-510n coupled to a system memory 520 via an input/output (I/O) interface 530. Computer system 500 further includes a network interface 540 coupled to I/O interface 530, and one or more input/output devices 550, such as cursor control device 560, keyboard 570, and display(s) 580. In various embodiments, any of the components may be utilized by the system to receive user input described above. In various embodiments, a user interface may be generated and displayed on display 580. In some cases, it is contemplated that embodiments may be implemented using a single instance of computer system 540, while in other embodiments multiple such systems, or multiple nodes making up computer system 500, may be configured to host different portions or instances of various embodiments. For example, in one embodiment some elements may be implemented via one or more nodes of computer system 500 that are distinct from those nodes implementing other elements. In another example, multiple nodes may implement computer system 500 in a distributed manner.

In different embodiments, computer system 500 may be any of various types of devices, including, but not limited to, a personal computer system, desktop computer, laptop, notebook, tablet or netbook computer, mainframe computer system, handheld computer, workstation, network computer, a camera, a set top box, a mobile device, a consumer device, video game console, handheld video game device, application server, storage device, a peripheral device such as a switch, modem, router, or in general any type of computing or electronic device.

In various embodiments, computer system 500 may be a uniprocessor system including one processor 510, or a multiprocessor system including several processors 510 (e.g., two, four, eight, or another suitable number). Processors 510 may be any suitable processor capable of executing instructions. For example, in various embodiments processors 510 may be general-purpose or embedded processors implementing any of a variety of instruction set architectures (ISAs). In multiprocessor systems, each of processors 510 may commonly, but not necessarily, implement the same ISA.

System memory 520 may be configured to store program instructions 522 and/or data 532 accessible by processor 510. In various embodiments, system memory 520 may be implemented using any suitable memory technology, such as static random-access memory (SRAM), synchronous dynamic RAM (SDRAM), nonvolatile/Flash-type memory, or any other type of memory. In the illustrated embodiment, program instructions and data implementing any of the elements of the embodiments described above may be stored within system memory 520. In other embodiments, program instructions and/or data may be received, sent or stored upon different types of computer-accessible media or on similar media separate from system memory 520 or computer system 500.

In one embodiment, I/O interface 530 may be configured to coordinate I/O traffic between processor 510, system memory 520, and any peripheral devices in the device, including network interface 540 or other peripheral interfaces, such as input/output devices 550. In some embodiments, I/O interface 530 may perform any necessary protocol, timing or other data transformations to convert data signals from one component (e.g., system memory 520) into a format suitable for use by another component (e.g., processor 510). In some embodiments, I/O interface 530 may include support for devices attached through various types of peripheral buses, such as a variant of the Peripheral Component Interconnect (PCI) bus standard or the Universal Serial Bus (USB) standard, for example. In some embodiments, the function of I/O interface 530 may be split into two or more separate components, such as a north bridge and a south bridge, for example. Also, in some embodiments some or all of the functionality of I/O interface 530, such as an interface to system memory 520, may be incorporated directly into processor 510.

Network interface 540 may be configured to allow data to be exchanged between computer system 500 and other devices attached to a network (e.g., network 590), such as one or more external systems or between nodes of computer system 500. In various embodiments, network 590 may include one or more networks including but not limited to Local Area Networks (LANs) (e.g., an Ethernet or corporate network), Wide Area Networks (WANs) (e.g., the Internet), wireless data networks, some other electronic data network, or some combination thereof. In various embodiments, network interface 540 may support communication via wired or wireless general data networks, such as any suitable type of Ethernet network, for example: via digital fiber communications networks: via storage area networks such as Fiber Channel SANs, or via any other suitable type of network and/or protocol.

Input/output devices 550 may, in some embodiments, include one or more display terminals, keyboards, keypads, touchpads, scanning devices, voice or optical recognition devices, or any other devices suitable for entering or accessing data by one or more computer systems 500. Multiple input/output devices 550 may be present in computer system 500 or may be distributed on various nodes of computer system 500. In some embodiments, similar input/output devices may be separate from computer system 500 and may interact with one or more nodes of computer system 500 through a wired or wireless connection, such as over network interface 540.

In some embodiments, the illustrated computer system may implement any of the operations and methods described above, such as the methods illustrated by the flowchart of FIG. 4. In other embodiments, different elements and data may be included.

Those skilled in the art will appreciate that computer system 500 is merely illustrative and is not intended to limit the scope of embodiments. In particular, the computer system and devices may include any combination of hardware or software that can perform the indicated functions of various embodiments, including computers, network devices, Internet appliances, PDAs, wireless phones, pagers, and the like. Computer system 500 may also be connected to other devices that are not illustrated, or instead may operate as a stand-alone system. In addition, the functionality provided by the illustrated components may in some embodiments be combined in fewer components or distributed in additional components. Similarly, in some embodiments, the functionality of some of the illustrated components may not be provided and/or other additional functionality may be available.

Those skilled in the art will also appreciate that, while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 500 may be transmitted to computer system 500 via transmission media or signals such as electrical, electromagnetic, or digital signals, conveyed via a communication medium such as a network and/or a wireless link. Various embodiments may further include receiving, sending or storing instructions and/or data implemented in accordance with the foregoing description upon a computer-accessible medium or via a communication medium. In general, a computer-accessible medium may include a storage medium or memory medium such as magnetic or optical media, e.g., disk or DVD/CD-ROM, volatile or non-volatile media such as RAM (e.g., SDRAM, DDR, RDRAM, SRAM, and the like), ROM, and the like.

The methods described herein may be implemented in software, hardware, or a combination thereof, in different embodiments. In addition, the order of methods may be changed, and various elements may be added, reordered, combined, omitted or otherwise modified. All examples described herein are presented in a non-limiting manner. Various modifications and changes may be made as would be obvious to a person skilled in the art having benefit of this disclosure. Realizations in accordance with embodiments have been described in the context of particular embodiments. These embodiments are meant to be illustrative and not limiting. Many variations, modifications, additions, and improvements are possible. Accordingly, plural instances may be provided for components described herein as a single instance. Boundaries between various components, operations and data stores are somewhat arbitrary, and particular operations are illustrated in the context of specific illustrative configurations. Other allocations of functionality are envisioned and may fall within the scope of claims that follow. Finally, structures and functionality presented as discrete components in the example configurations may be implemented as a combined structure or component. These and other variations, modifications, additions, and improvements may fall within the scope of embodiments as defined in the claims that follow.

In the foregoing description, numerous specific details, examples, and scenarios are set forth in order to provide a more thorough understanding of the present disclosure. It will be appreciated, however, that embodiments of the disclosure may be practiced without such specific details. Further, such examples and scenarios are provided for illustration, and are not intended to limit the disclosure in any way. Those of ordinary skill in the art, with the included descriptions, should be able to implement appropriate functionality without undue experimentation.

References in the specification to “an embodiment,” etc., indicate that the embodiment described may include a particular feature, structure, or characteristic, but every embodiment may not necessarily include the particular feature, structure, or characteristic. Such phrases are not necessarily referring to the same embodiment. Further, when a particular feature, structure, or characteristic is described in connection with an embodiment, it is believed to be within the knowledge of one skilled in the art to affect such feature, structure, or characteristic in connection with other embodiments whether or not explicitly indicated.

Embodiments in accordance with the disclosure may be implemented in hardware, firmware, software, or any combination thereof. Embodiments may also be implemented as instructions stored using one or more machine-readable media, which may be read and executed by one or more processors. A machine-readable medium may include any mechanism for storing or transmitting information in a form readable by a machine (e.g., a computing device or a “virtual machine” running on one or more computing devices). For example, a machine-readable medium may include any suitable form of volatile or non-volatile memory.

Modules, data structures, and the like defined herein are defined as such for ease of discussion and are not intended to imply that any specific implementation details are required. For example, any of the described modules and/or data structures may be combined or divided into sub-modules, sub-processes or other units of computer code or data as may be required by a particular design or implementation.

In the drawings, specific arrangements or orderings of schematic elements may be shown for ease of description. However, the specific ordering or arrangement of such elements is not meant to imply that a particular order or sequence of processing, or separation of processes, is required in all embodiments. In general, schematic elements used to represent instruction blocks or modules may be implemented using any suitable form of machine-readable instruction, and each such instruction may be implemented using any suitable programming language, library, application-programming interface (API), and/or other software development tools or frameworks. Similarly, schematic elements used to represent data or information may be implemented using any suitable electronic arrangement or data structure. Further, some connections, relationships or associations between elements may be simplified or not shown in the drawings so as not to obscure the disclosure.

Any numerical values recited herein are exemplary, are not to be considered limiting, and include ranges therebetween, and can be inclusive or exclusive of the endpoints. Optional included ranges can be from integer values therebetween, at the order of magnitude recited or the next smaller order of magnitude. For example, if the lower range value is 0.1, optional included endpoints can be 0.2, 0.3, 0.4 . . . 1.1, 1.2, and the like, as well as 1, 2, 3 and the like: if the higher range is 10, optional included endpoints can be 7, 6, and the like, as well as 7.9, 7.8,and/or the like.

Although some embodiments have been discussed above, other implementations and applications are also within the scope of the following claims. Although various embodiments herein have been referred to with particularity, it is to be understood that these embodiments are merely illustrative of the principles and applications of the various embodiments. It is therefore to be understood that modifications may be made to the illustrative embodiments and other embodiments may be devised without departing from the spirit and scope of the present disclosure.

Publications and references, including but not limited to patents and patent applications, cited in this specification are herein incorporated by reference in their entireties as if each individual publication or reference were specifically and individually fully set forth herein. Any patent application to which this application claims priority is also incorporated by reference herein in the manner described above for publications and refer.

1. A wearable system for tracking hand and wrist motion, the system comprising
a wearable motion pod configured to be worn on a hand or wrist, the motion pod including:
a sensor configured to output an orientation and movement measurement of the hand or wrist,
a haptic feedback device configured to provide sensory feedback to the hand or wrist,
a processor in communication with the sensor and the haptic feedback device, the processor configured to determine whether the hand is in a neutral position based on the orientation and movement measurement of the hand or wrist and output a haptic feedback signal to the haptic feedback device based on the determination of whether the hand is in a neutral position; and
a memory in communication with the processor and configured to record measurements from the sensors as measurement data.

2. The system according to claim 1, wherein the processor is configured to determine an amount of time that the hand is not in the neutral position out of a predetermined measurement time window.

3. The system according to claim 1, wherein the processor is configured to determine a number of times the haptic feedback signal is output over a certain period of time.

4. The system according to claim 1, wherein the neutral position is within a range of 0 to 30% of a maximum range of motion of the hand.

5. The system according to claim 1, further comprising personal protective equipment to which the pod is attached.

6. The system according to claim 1, wherein the sensory feedback includes at least one of a visual, audio, or tactile feedback.

7. The system according to claim 1, wherein the sensor includes a plurality of sensors including a gyroscope and an accelerometer.

8. The system according to claim 7, wherein the plurality of sensors includes at least one of an altimeter, a temperature sensor, magnetometer, or a relative humidity sensor.

9. The system according to claim 1, further comprising:
a charging tower configured to connect to and charge at least one pod.

10. The system according to claim 1, wherein the pod further includes:
a communication module configured to communicate with a network to transmit the measurement data for processing.

11. The system according to claim 10, further comprising a data processing system configured to receive the measurement data from the network, perform data analysis on the measurement data, and generate reports based on the analysis.

12. A method for tracking hand and wrist motion, the method comprising:
i) measuring an orientation and movement of a hand or wrist of a user;
ii) determining from the measuring in i) whether the hand is in a neutral position:
iii) incrementing a compliance time if the hand is in a neutral position and incrementing a non-compliance time if the hand is not in a neutral position:
determining whether a measurement time window has expired:
if the measurement time window has not expired, repeating i), ii), and iii), and if the time window has expired, determining whether a total non-compliance time is greater than a threshold; and
if the total non-compliance time is greater than the threshold, generating an alert.

13. The method according to claim 12, wherein determining whether the hand is in a neutral position includes comparing measurements of the orientation and movement of the hand or wrist against a range of measurements defining a neutral zone, and determining that the hand is in a neutral position if the measurements of the hand or wrist are within the range of measurements defining the neutral zone and determining that the hand is not in a neutral position if the measurements of the hand or wrist are not within the range of measurements defining the neutral zone.

14. The method according to claim 13, further comprising:
updating at least one of the measurement time window, the threshold, or the range of measurements defining the neutral zone based upon an analysis of a total number of alerts generated over a certain period of time greater than the measurement time window.

15. A wearable pod configured to be worn on a hand or wrist, the pod including:
a plurality of sensors configured to output an orientation and movement measurement of the hand or wrist, the plurality of sensors including an accelerometer, and a gyroscope,
a haptic feedback device configured to provide sensory feedback to the hand or wrist;
a processor in communication with the sensor and the haptic feedback device, the processor configured to determine whether the hand is in a neutral position based on the orientation and movement measurement of the hand or wrist and output a haptic feedback signal to the haptic feedback device based on the determination of whether the hand is in a neutral position;
a memory in communication with the processor and configured to record measurements from the sensors as measurement data;
a power supply configured to supply power to the pod; and
a communication module configured to communicate with a network to transmit the measurement data for processing.
